(19) United States
(12) Patent Application Publication
(10) Pub. No.: US 2009/0082787 A1
(43) Pub. Date: Mar. 26, 2009

(54) Suturing Device

(76) Inventor: Ah San Pang, Singapore (SG)

Correspondence Address: EDELL, SHAPIRO & FINNAN, LLC, 1901 RESEARCH BLVD., SUITE 400, ROCKVILLE, MD, 20850-3164, US

(21) Appl. No.: 12/127,865

(22) Filed: May 28, 2008

Related U.S. Application Data
(63) Continuation of application No. PCT/SG2007/000376, filed on Nov. 6, 2007.

(30) Foreign Application Priority Data
Nov. 6, 2006 (SG) 200607706-9

(51) Int. Cl. A61B 17/12; A61B 17/10

(52) U.S. Cl. 606/144; 606/139

(57) ABSTRACT

A suturing device is disclosed. The suturing device comprises a shaft with a proximal end and a distal end and a passage extending therebetween disposed on a longitudinal axis. A rod is disposed within the shaft passage and is capable of moving axially along the longitudinal axis. A loop-forming opening extends partly along the shaft and into the shaft passage, and is disposed proximal to the distal end. A stop is disposed between the rod and the distal end. A resiliently flexible wire is coupled to the rod and to the stop. In use, movement of the rod towards the distal end compresses the wire against the stop to form a suture-receiving loop extending from the loop-forming opening and wherein movement of the rod towards the proximal end retracts the suture-receiving loop from the loop-forming opening to move the wire into the shaft.

### TECHNICAL FIELD

The present invention generally relates to a suturing device for use in a surgical procedure.

### BACKGROUND

During operations, stitches or sutures are applied to the wound areas in order to close up the wound to facilitate healing. These sutures are applied to the affected body areas with the aid of surgical needles.

Typically, there are two types of surgical needles that are used with sutures. The first type is known as the “traumatic type” whereby the suture thread is threaded through the eye of the surgical needle. This mechanism is similar to a threaded sewing needle whereby the thread is threaded through the eye of the needle. Generally, it is often inconvenient to use traumatic needles because there is a need for the surgeon to manually thread the suture through the eye of the traumatic needle before stitching. This can lead to fraying or damage of the suture in cases of repeated threading or incessant rubbing of the suture thread with the eye. Depending on the size and type of the traumatic needle and suture, the eye of the needle may be small and a certain degree of trail and error is required before the suture is threaded through the eye. Furthermore, due to the open nature of the eye, the suture may become unthreaded prematurely.

The second type of surgical needle is known as the “atraumatic type” whereby the needle is eyeless. Typically, the suture is pre-attached and packaged with an atraumatic needle such that the suture is along the longitudinal axis of the needle and extends from it. The suture may be permanently attached to the needle or may be detached from the needle by physical action. Due to the pre-attachment of the suture to the needle, there are a number of commercially available combinations of sutures with the corresponding needle sizes. However, should the surgeon require a particular combination of needle and suture that is not commercially available, he/she would have to compromise on using a different sized suture or needle. Moreover, due to the integrated combination of suture and surgical needle, such a system is inflexible should a need to detach the suture prematurely from the surgical needle arise.

There is a need to provide a suturing device that overcomes, or at least ameliorates, one or more of the disadvantages described above.

### SUMMARY

According to a first aspect of the invention, there is provided a suturing device comprising:
a shaft with a proximal end and a distal end and a passage extending therebetween disposed on a longitudinal axis;
a rod disposed within said shaft passage and capable of moving axially along said longitudinal axis;
a loop-forming opening extending partly along said shaft and into said shaft passage, and being disposed between said proximal and said distal ends;
a stop disposed between said rod and said distal end; and
a resiliently flexible wire coupled to said rod and to said stop wherein movement of said rod towards said distal end compresses said wire against said stop to form a suture-receiving loop extending from said loop-forming opening and wherein movement of said rod towards said proximal end retracts said suture-receiving loop from said loop-forming opening to move said wire into said shaft.

Advantageously, the loop formed from said wire allows part of a suture to be inserted therethrough and closure of the loop allows the suture to be captured by the suturing device.

There may be provided sealing means in said shaft passage for preventing fluid, typically gas, present within said shaft passage from being transmitted from said proximal end. In one embodiment, the sealing means is at least a portion of the rod being dimensioned to sealingly connect with the shaft passage. For example, in embodiments where the shaft passage is circular in cross-section at a normal angle to the longitudinal axis, the diameter of the rod may be such that the rod abuts the shaft passage to prevent the egress of gas but slightly smaller than the shaft passage to allow the rod to move axially within the shaft passage. In other embodiments, the rod may be dimensioned such that the outer diameter of the rod is smaller than the inner diameter of the shaft but the end of the shaft passage may have a portion which is dimensioned to abut with the shaft to prevent the egress of gas from the proximal end while still allowing the rod to move axially therein. In yet another embodiment, the sealing means may not be the rod itself but may be located within the shaft passage, such as an o-ring mounted on the end of the rod, for preventing the transmission of gas towards the proximal end of the shaft. During endoscopic examination, the viscus which is being examined must remain distended with air. A collapsed viscus cannot be properly examined. During endoscopic suturing, it is even more important that the viscus does not collapse. If too much gas escapes through the suturing device, suturing becomes laborious, if not impossible. Advantageously, when the suturing device is being used to suture a patient's viscus, gasses located within the viscus may be preventing from being transmitted from the proximal end while the loop-forming opening is in fluid communication with the viscus. Accordingly, the sealing means ensures that the viscus remains inflated by substantially preventing the egress of the gas from the viscus during suturing.

In one embodiment, the loop-forming opening is adjacent or proximal to the distal end of the shaft.

The suturing device may further comprise a bias for urging the rod towards either the distal end or the proximal end.

The suturing device may further comprise a pointed head disposed at the distal end of the shaft. In one embodiment, the pointed head is a puncture tip for puncturing the tissue of an animal. Exemplary forms of a puncture tip can be selected from the group consisting of a solid trocar point, a solid bevel point and a closed pencil point.

Advantageously, the stop is moveable within the shaft passage. In one embodiment, the stop is an axially movable second rod disposed within said shaft. In another embodiment, the stop is a ball disposed within said shaft and moveable along said longitudinal axis. In yet another embodiment, the stop is a stationary rod disposed at the distal end of the shaft. In a further embodiment, the stop is a wall disposed at the distal end of the shaft.

In one embodiment, the rod, resiliently flexible wire and the stop are formed from a unitary member.

The stop is dimensioned such that it does not escape from the loop-forming opening. Advantageously, the stop functions to grasp the suture firmly within the shaft passage so that the suture does not disengage from the shaft.

In one embodiment, the resiliently flexible wire may be laser welded to the rod and the stop.

The suturing device may further comprise a latch for locking said rod to inhibit movement of said rod by said bias towards either said distal end or said proximal end. Advantageously, the latch locks the rod to ensure that it does not move within the shaft when the wire is in the form of the loop or when the loop has been retracted and the wire resides in the shaft. Hence, the latch locks the caught suture within the shaft as the suturing device is moved through a tissue. The latch also releases the caught suture from the shaft after the suturing device has moved through the tissue. Advantageously, the locked position allows a surgeon to concentrate on threading the suture. The suturing device may comprise a button coupled to said latch and disposed at the proximal end of said shaft for moving said rod axially within said shaft. Advantageously, the button allows the surgeon to move the wire to form the hook or to deform the hook, simply by depressing the button. The latch may be configured such that depressing the button once will form the wire into a loop and then while thereafter depressing the button again will deform the wire into the shaft. The button may be an end knob as discussed further below.

In one embodiment, the shaft is substantially cylindrical. Advantageously, a cylindrical shaped shaft has no sharp edges thereon to facilitate passage of the shaft into the tissue during a suturing procedure.

The axially moveable rod may be biased towards said proximal end of the shaft. In one embodiment, the bias is a spring disposed within the shaft. It should be realized that other embodiments may use different biasing means, such as a compressible rubber member.

The bias may be capable of controlling the size of the suture-receiving loop. The biasing of the rod towards the proximal end may allow firm grasping of the suture within the shaft passage.

In use, the surgeon exerts a force on the button to compress the wire and form it into a loop. In one embodiment, a spring bias provides a resistive force to the compressive force exerted by the surgeon when he/she depresses the button. The resistive force provided by the spring bias allows the surgeon to have better control over movement of the moveable rod within the shaft and hence the size of the loop. Eventually, as will be described further below, if a sufficient force is exerted by the surgeon, the bias force is overcome to form a maximum sized loop and the latch holds the loop in place. The surgeon then threads a suture through the loop. After which, the surgeon depresses the button and the rod moves towards the proximal end of the shaft by the action of the spring bias. The movement by the rod retracts the wire and the associated suture back into the shaft passage such that the suture is grasped firmly within the shaft passage.

In one embodiment, the loop-forming opening is in the form of a narrow slit, with the longitudinal axis of said narrow slit extending parallel to the longitudinal axis of said shaft. The narrow slit may be rectangular-shaped. Advantageously, the side edges of the rectangular shaped opening assist in the formation of the loop by providing an abutting surface in which the loop is compressed to ‘pop-out’ of the opening.

According to a second aspect, there is provided a method of manipulating a suture in a tissue by releasing and capturing the suture within a body cavity, the suture having a first and a second part, comprising the steps of:

providing a suturing device as defined in the first aspect above;

positioning the suture with at least the first part of the suture on a first side of the tissue;

using the suturing device to engage and pass the first part of the suture through the tissue to the second side of the tissue at one point, leaving the second part of the suture on the first side of the tissue, thereby positioning the suture with the two parts of the suture on opposite sides of the tissue;

releasing the first part of the suture and withdrawing the suturing device through the tissue at said one point, and passing it through the tissue at a second point spaced from said one point; and

forming the suture-receiving loop extending from said loop-forming opening by moving said rod towards the distal end, capturing the first part of the suture in the loop of the suturing device, closing the loop by moving the rod towards the proximal end, and drawing the first part of the suture through the tissue from the second side to the first side at the second point, so as to enable forming a stitch through the tissue.

In one embodiment, manipulating the suture in the tissue occurs in a gastropexy suturing procedure.

A third aspect provides a kit for performing a suturing procedure comprising:

a suturing device as define in the first aspect above; and

instructions for using the suturing device in the suturing procedure.

### Definitions

The following words and terms used herein shall have the meaning indicated:

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

### BRIEF DESCRIPTION OF DRAWINGS

accompanying drawings illustrate a disclosed embodiment and serves to explain the principles of the disclosed embodiment. It is to be understood, however, that the drawings are designed for purposes of illustration only, and not as a definition of the limits of the invention.

FIG. 1A is a perspective diagram of a suturing device in the ‘closed’ position.

FIG. 1B is a perspective diagram of the suturing device of FIG. 1A in the ‘open’ position.

FIG. 1C is a side view of the suturing device of FIG. 1B in the ‘open’ position.

FIG. 2A is a cross sectional side view of the suturing device of FIG. 1A in the ‘closed’ position according to one embodiment.

FIG. 2B is a cross sectional side view of the suturing device of FIG. 1C in the ‘open’ position according to one embodiment.

FIG. 3A is a cross sectional side view of the suturing device of FIG. 1A in the ‘closed’ position according to another embodiment.

FIG. 3B is a cross sectional side view of the suturing device of FIG. 1C in the ‘open’ position according to another embodiment.

FIG. 4A is a perspective diagram of a suturing device controlled by an actuating mechanism in the ‘closed’ position.

FIG. 4B is a perspective diagram of a suturing device controlled by an actuating mechanism in the ‘open’ position.

FIG. 5A is a cross sectional side view of the suturing device of FIG. 4A in the ‘closed’ position.

FIG. 5B is a cross sectional side view of the suturing device of FIG. 4B in the ‘open’ position.

FIG. 6A is an expanded cross sectional diagram of the actuating mechanism of FIG. 4A in the ‘closed’ position.

FIG. 6B is an expanded cross sectional diagram of the actuating mechanism of FIG. 4B in the ‘open’ position.

### DETAILED DISCLOSURE OF EMBODIMENTS

Exemplary, non-limiting embodiments of a suturing device will now be disclosed.

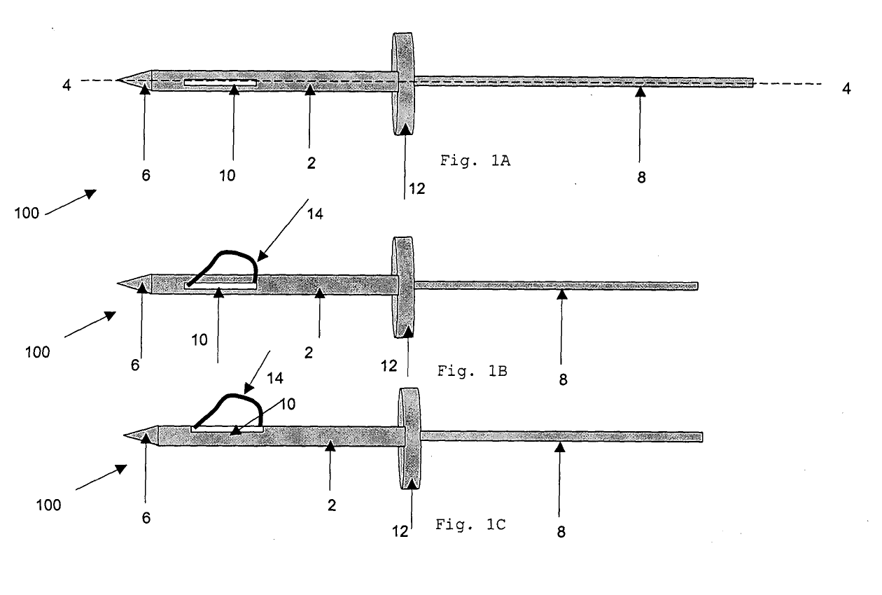

FIG. 1A and FIG. 1B are perspective diagrams of a suturing device 100 in the ‘closed’ position and ‘open’ position respectively. The suturing device 100 has a hollow shaft 2 with a proximal end and a distal end and a passage extending therebetween disposed on a longitudinal axis 4 as denoted by the dashed line.

A pointed head in the form of a pointed needle 6 is disposed on the distal end and a rod 8 is disposed at the proximal end. The needle 6 acts as a puncturing device to allow passage of the suturing device 100 into the body during operations which require the use of a suture. The rod 8 extends into the shaft passage and is dimensioned such that the rod 8 is capable of moving axially along the longitudinal axis 4.

A loop-forming opening 10 extends partly along shaft 2 and into the shaft passage. The loop-forming opening 10 is disposed proximal to the pointed needle 6. A holding support 12 is disposed at the proximal end of the shaft 2. A hole (not shown) is disposed in the holding support 12 to allow movement of rod 8 into shaft 2. A resiliently flexible wire 14 protrudes from the loop-forming opening 10 as seen in FIG. 1B. The resiliently flexible wire 14 forms a loop which allows a suture to be threaded through.

Referring now to FIG. 1C, there is provided a side view of the suturing device of FIG. 1B. It can be seen that the loop-forming opening 10 extends into the shaft passage. Furthermore, the loop-forming opening 10 is in the form of a narrow slit that extends parallel to the longitudinal axis 4 of the shaft 2.

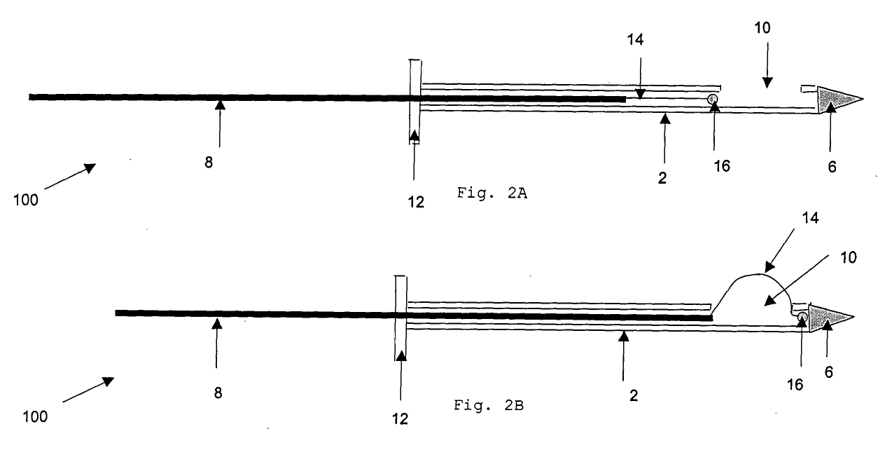

FIG. 2A and FIG. 2B are cross sectional side view diagrams of the suturing device 100 of FIG. 1A and FIG. 1B in the ‘closed’ position and ‘open’ position respectively. As seen, the shaft 2 is hollow and the bore of the shaft 2 defines a passage which allows for movement of the rod 8. The rod 8 is connected to the resiliently flexible wire 14 and a stop in the form of a ball 16. The resiliently flexible wire 14 is laser welded to both the rod 8 and the ball 16. The ball 16 is dimensioned to be bigger than the dimensions of the loop-forming opening 10 such that the ball 16 is prevented from exiting the shaft passage through the loop-forming opening 10.

In use, the rod 8 is manually pushed into the shaft passage such that the rod 8, resiliently flexible wire 14 and ball 16 moves towards the distal end along the longitudinal axis. As the ball 16 encounters the end of the shaft 2, the resiliently flexible wire 14 is forced out of the loop-forming hole 10 by the continued movement of the rod 8. The resiliently flexible wire 14 forms a loop which allows for a suture to be threaded through. The suturing device 100 is now in the ‘open’ position as seen in FIG. 2B.

In order to engage the suture firmly with the suturing device 100, the rod 8 is manually moved in the direction towards the proximal end along the longitudinal axis. The resiliently flexible wire 14 retracts into the shaft passage and engages the suture within the shaft passage. The suturing device 100 is now in the ‘closed’ position as seen in FIG. 2A. The suture inside the shaft passage does not dissociate from the suturing device 100 as there is no opening in the ‘closed’ position.

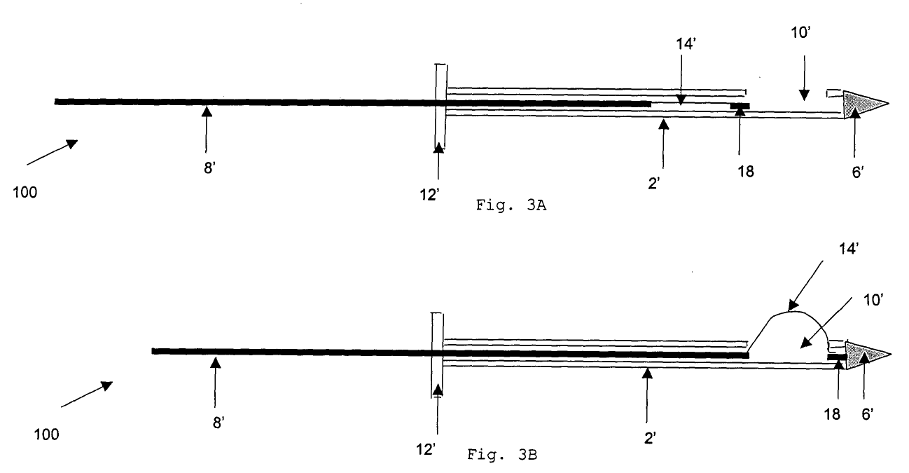

FIG. 3A and FIG. 3B are cross sectional side view diagrams of a suturing device 100′ in the ‘closed’ position and ‘open’ position respectively. FIG. 3A and FIG. 3B are similar to the suturing device 100 of FIG. 2A and FIG. 2B and are denoted by the same reference numerals but with a prime (′) symbol.

Here, a stop in the form of a second rod 18 is connected to the resiliently flexible wire 14′ instead of a ball 16 as shown in FIG. 2A and FIG. 2B. The suturing device 100′ works in the same way as discussed above.

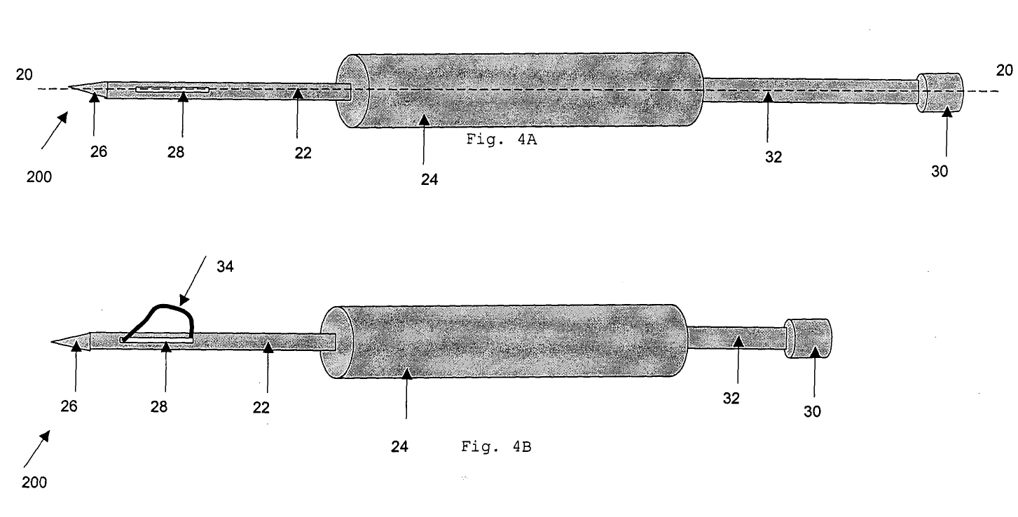

FIG. 4A and FIG. 4B are perspective diagrams of a suturing device 200 according to another embodiment in the ‘closed’ position and ‘open’ position respectively. The suturing device 200 has a hollow shaft 22 with a proximal end and a distal end and a passage extending therebetween disposed on a longitudinal axis 20 as denoted by the dashed line.

A pointed needle 26 is disposed on the distal end and a actuating mechanism 24 is disposed at the proximal end. The needle 26 acts as a puncturing device to allow passage of the suturing device 200 into the body during operations which require the use of a suture. A release mechanism 32 connected to a button in the form of an end knob 30 extends into the actuating mechanism 24.

The movement of the release mechanism 32 and end knob 30 into the actuating mechanism 24 allows the resiliently flexible wire 34 to be protruded from the loop-forming hole 28 as will be discussed further below.

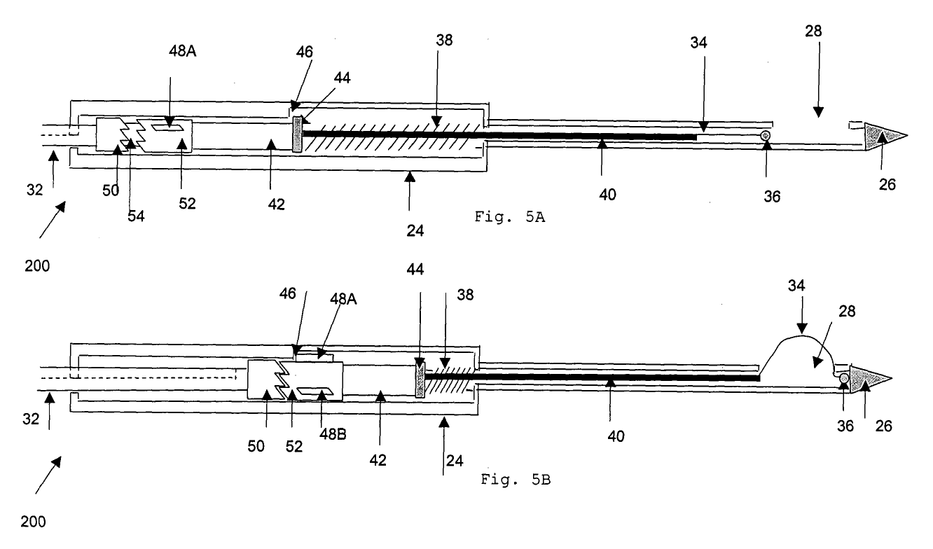

Referring to FIG. 5A and FIG. 5B, there are shown cross sectional side view diagrams of the suturing device 200 of FIG. 4A and FIG. 4B respectively. The actuating mechanism 24 houses a bias in the form of a spring bias 38, a slide cylinder 42, a first cam 50, a second cam 52 and a cam ring 54.

The rod 40 extends into a hole (not shown) disposed within the slide cylinder 42 at the rod support 44.

Similarly, the release mechanism 32 extends into a hole (not shown) in the first cam 50. The first cam 50 and second cam 52 are loosely connected to the cam ring 54 such that they are freely rotatable about the cam ring 54.

The slide cylinder 42 is attached to the second cam 52.

Three notches, of which one is shown in FIG. 5A by the reference numeral 48A and two are shown in FIG. 5B by the reference numeral 48A, 48B, are disposed on the surface of the second cam 52.

The notches 48A, 48B are designed such that any of them can be caught by the groove 46 that is built into the wall of the actuating mechanism 24. It should be appreciated that the number of grooves in the walls of the actuating mechanism 24 corresponds to the number of notches (48A, 48B).

In use, as the release mechanism 32 moves towards the distal end by the depressing of the end knob (not shown), the jagged edges of the first cam 50 and the jagged edges of the second cam 52 engages with each other. The movement of the slide cylinder 42 towards the distal end compresses the spring bias 38 and causes movement of the rod 40, resiliently flexible wire 34 and ball 36 towards the needle 26 to form a loop that protrudes from the loop-forming hole 28. As the second cam 52 rotates, the notch 48A catches and latches onto the groove 46. The notch 48A acts as a latch to prevent movement of the rod 40 and hence the loop when the surgeon is threading the suture (not shown). The suturing device 200 with the formed loop is seen in FIG. 5B.

When the end knob (not shown) is depressed a second time, the rotation of the second cam 52 rotates the notch 48A away from the groove 46. This allows the bias action of the spring bias 38 to push the slide cylinder 42, first cam 50, cam ring 54 and second cam 52 towards the proximal end. The loop retracts into the shaft passage and grasps the suture (not shown) firmly within the shaft passage as shown in FIG. 5A.

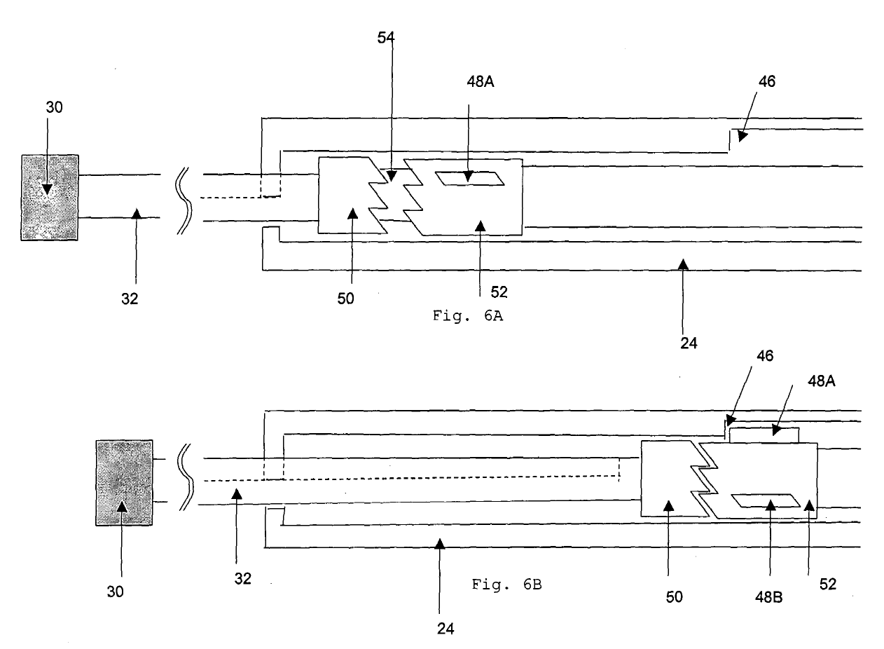

FIG. 6A and FIG. 6B are expanded cross sectional diagrams of the actuating mechanism corresponding to FIG. 5A and FIG. 5B respectively. In use, the suturing device (200) of FIG. 4 is used for adding a gastropexy during a pull-through percutaneous endoscopic gastrosomy (PEG) procedure.

After the PEG procedure had been completed, the suturing device (200) was used to anchor the stomach wall to the abdominal wall at three cardinal points.

The suturing device (200) was first threaded with a suture. This was carried out by first depressing the release mechanism (32) via the end knob (30) such that the loop as defined by the resiliently flexible wire (34) forms. The loop allows the suture to be threaded through it. In order to secure the suture to the suturing device (200), the release mechanism (32) was depressed again such that the resiliently flexible wire (34) retracts into the opening (28) and holds the suture firmly within the suturing device (200).

A tiny stab incision was made in the skin. Through this incision, the suturing device (200) was used to puncture the abdominal and stomach walls. As the suturing device (200) enters into the stomach cavity, one end of the threaded suture passes into the stomach cavity along with the suturing device (200) with the other end left outside the body. An endoscope was used to visualize the stomach cavity and to aid in the creation of the gastropexy. A standard endoscopic biopsy forceps was used to remove the suture from the suturing device (200). The suturing device (200) was then removed. Following which, the suturing device (200) was used to make a second puncture through the same incision but slightly away from the first puncture.

Here, the release mechanism (32) is depressed again such that the loop forms in the stomach cavity. By looking through the endoscope, the position of the suturing device (200) and the loop could be determined. The size of the loop could be controlled, if needed, by controlling the movement of the release mechanism (32). Using the endoscopic biopsy forceps, the suture was threaded though the loop of the suturing device (200) at the second puncture position. The release mechanism (32) was then depressed such that the resiliently flexible wire (34) retracts into the opening (28), grasping the suture firmly into the shaft passage.

The suturing device (200) was then withdrawn a second time, bringing the end of the suture back outside the body. The two ends of the suture were tied together such that the stomach wall was anchored to the abdominal wall.

As the suturing device (200) was in the ‘closed’ position during puncture and removal from the body, trauma to the body tissues was minimized. In addition, due to the large loop that was formed in the ‘open’ position, the loop could be easily visualized by the endoscope.

Where the suturing device (200) is used to suture a stomach, for each cardinal point there will be two puncturing passes of the suturing device (200). If the second puncturing pass is made at a slight angle (ie of less than about 60 degrees) to the first puncture pass, it is possible to keep the puncture holes on the stomach side close together while the puncture holes on the abdominal wall side are spaced apart.

The process was repeated to securely anchor the stomach to the abdominal walls at the remaining two cardinal points.

It is to be appreciated that the process can be repeated to anchor the stomach to the abdominal walls at more than three cardinal points.

There is also provided a kit for performing a suturing procedure. The kit comprises a suturing device (100, 200) and instructions for using the suturing device (100, 200) in a surgical or suturing procedure such as a gastropexy suturing procedure, an urinary cystopexy suturing procedure or a sigmoidopexy suturing procedure.

### Applications

The suturing device as disclosed herein is suitable for use during an endoscopic gastrostomy procedure whereby a gastropexy is to be added. Furthermore, it may be used for a urinary cystopexy and a sigmoidopexy.

Advantageously, the size of the loop can be manipulated as desired so that the surgeon is able to thread a suture through the loop. This is possible even when an endoscope is used to guide the surgeon during the threading process. The suturing device may provide surgeons with greater control over the threading process.

Advantageously, the loop formed in the suturing device can be variable and this provides the surgeon with greater flexibility when choosing a suitable type and size of suture.

Advantageously, the variable size of the loop allows surgeons to thread the suture through in the first attempt. Hence, the suturing device may minimize any damage to the suture such as fraying as a result of repeated tries at threading. Damaged sutures may result in lesser integrity and may break easily.

The suturing device may grasp the suture firmly within the shaft such that the suture does not dissociate from the suturing device as it moves through a tissue. Importantly, this ensures that the suture is not lost within a tissue and this enables greater control with regards to the positioning of a suture at certain sites of operation.

Advantageously, the disclosed suturing device may be used to suture a stomach, wherein the puncture holes on the stomach side are relatively close together while the puncture holes on the abdominal wall side are spaced apart.

Advantageously, the disclosed suturing device may have a sealing means in the form of the diameter of the rod which abuts internal the walls of the shaft passage to prevent the egress of gas from the shaft to the proximal end of the shaft. Hence, the disclosed device can for example be used to suture a viscus without releasing gas from the viscus and causing deflation thereof.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A suturing device comprising:
a shaft with a proximal end and a distal end and a passage extending therebetween disposed on a longitudinal axis;
a rod disposed within said shaft passage and capable of moving axially along said longitudinal axis;
a loop-forming opening extending partly along said shaft and into said shaft passage, and being disposed between said proximal and said distal ends;
a stop disposed between said rod and said distal end; and
a resiliently flexible wire coupled to said rod and to said stop wherein movement of said rod towards said distal end compresses said wire against said stop to form a suture-receiving loop extending from said loop-forming opening and wherein movement of said rod towards said proximal end retracts said suture-receiving loop from said loop-forming opening to move said wire into said shaft.

2. A suturing device as claimed in claim 1, comprising a bias for urging said rod towards either said distal end or said proximal end.

3. A suturing device as claimed in claim 1 or claim 2, wherein said loop-forming opening is disposed adjacent to said distal end.

4. A suturing device as claimed in any one of the preceding claims comprising a sealing means disposed within said shaft passage for preventing fluid present within said shaft passage from being transmitted to said proximal end of said shaft.

5. A suturing device as claimed in claim 4, wherein the sealing means comprises at least a portion of the rod being dimensioned to abut the sidewalls of the shaft passage to form a fluid seal therewith while allowing said rod to move axially therein.

6. A suturing device as claimed in any one of the preceding claims, comprising a pointed head disposed at the distal end of said shaft.

7. A suturing device as claimed in any one of the preceding claims, wherein the stop is moveable within said shaft.

8. A suturing device as claimed in claim 7, wherein the stop is an axially moveable second rod disposed within said shaft.

9. A suturing device as claimed in claim 7, wherein the stop is a ball disposed within said shaft and moveable along said longitudinal axis.

10. A suturing device as claimed in any one of claims 1 to 6, wherein said stop is a stationary rod disposed at the distal end of said shaft.

11. A suturing device as claimed in any one of claims 1 to 6, wherein said stop is a wall disposed at the distal end of said shaft.

12. A suturing device as claimed in any one of the preceding claims, comprising a latch for locking said rod to inhibit movement of said rod by said bias towards either said distal end or said proximal end.

13. A suturing device as claimed in claim 12, comprising a button coupled to said latch and disposed at the proximal end of said shaft for moving said rod axially within said shaft.

14. A suturing device as claimed in any one of the preceding claims, wherein said rod, said resiliently flexible wire and said stop are formed from a unitary member.

15. A suturing device as claimed in any one of the preceding claims, wherein said shaft is substantially cylindrical.

16. A suturing device as claimed in any one of the preceding claims, comprising a bias to bias said rod towards said proximal end.

17. A suturing device as claimed in claim 16, wherein said bias is a spring.

18. A suturing device as claimed in claim 16, wherein said bias assists in controlling the size of said suture-receiving loop.

19. A suturing device as claimed in claim 6, wherein said pointed head forms a puncture tip for puncturing the tissue of an animal.

20. A suturing device as claimed in claim 19, wherein said puncture tip is in the form selected from the group consisting of a solid trocar point, a solid bevel point and a closed pencil point.

21. A suturing device as claimed in any one of the preceding claims, wherein said loop-forming opening is in the form of a narrow slit, with the longitudinal axis of said narrow slit extending parallel to the longitudinal axis of said shaft.

22. A method of manipulating a suture in a tissue by releasing and capturing the suture within a body cavity, the suture having a first and a second part, comprising the steps of:
providing a suturing device as claimed in any one of claims 1 to 21;
positioning the suture with at least the first part of the suture on a first side of the tissue;
using the suturing device to engage and pass the first part of the suture through the tissue to the second side of the tissue at one point, leaving the second part of the suture on the first side of the tissue, thereby positioning the suture with the two parts of the suture on opposite sides of the tissue;
releasing the first part of the suture and withdrawing the suturing device through the tissue at said one point, and passing it through the tissue at a second point spaced from said one point; and
forming the suture-receiving loop extending from said loop-forming opening by moving said rod towards said distal end, capturing the first part of the suture in the loop of said suturing device, closing the loop by moving said rod towards said proximal end, and drawing the first part of the suture through the tissue from the second side to the first side at said second point, so as to enable forming a stitch through the tissue.

23. A method as claimed in claim 22, wherein manipulating said suture in said tissue occurs in a suturing procedure selected from the group consisting of a gastropexy suturing procedure, an urinary cystopexy suturing procedure and a sigmoidopexy suturing procedure.

24. A kit for performing a suturing procedure comprising:
a suturing device as claimed in any one of claims 1 to 21; and
instructions for using said suturing device in said surgical procedure.

25. A kit as claimed in claim 24, wherein the instructions are for performing a suturing procedure selected from the group consisting of a gastropexy suturing procedure, an urinary cystopexy suturing procedure and a sigmoidopexy suturing procedure.
